(19) United States
(12) Patent Application Publication
(10) Pub. No.: US 2011/0137106 A1
(43) Pub. Date: Jun. 9, 2011

(54) Magnetic clip for ear acupuncture points

(76) Inventors: Chih-Kuo Liang, Yung-Kang City (TW); Jin-Chen Tsai, Yung-Kang City (TW); Chin-Yu Liu, Yung-Kang City (TW)

(21) Appl. No.: 12/591,967

(22) Filed: Dec. 7, 2009

(51) Int. Cl. A61N 2/00

(52) U.S. Cl. 600/15

(57) ABSTRACT

A magnetic clip for ear acupuncture points comprises a first clip body and a second clip body. An insulation cap is provided on one side of the first clip body, and then a first magnetic body is accommodated between the first clip body and the insulation cap. The second clip body is provided with a second magnetic body. The first magnetic body and the second magnetic body are coupled with lead wires respectively which in turn are both connected to an electric stimulator. The first magnetic body and the second magnetic body can sandwich the ear to be treated between them by magnetic force which is used simultaneously to conduct magnetic therapy on ear acupuncture points. The electric stimulator can also send electric current to the magnetic bodies through the lead wires, so as to conduct electric therapy via the stimulating probe.

### BACKGROUND OF THE INVENTION

1. Field of the Invention

The present invention relates to a magnetic clip for ear acupuncture points, particularly to a magnetic clip for ear acupuncture points which conducts magnetic therapy upon ear acupuncture points by magnetic force, and which provides required holding force so as to avoid drop-down of the clip.

2. Brief Description of Prior Art

A conventional electric therapy device is disclosed in a Taiwanese Patent No. 289260 entitled “ Ear Clip of Electronic Acupuncture for Ear Acupuncture Points” , in which the ear clip comprises: a primary clip pad, the upper end of which is formed with a clip portion, the clip portion being substantially an uncertain shape accommodation space for the positioning of an object therein, a through opening being formed at the side edge of the accommodation space; the lower end of the primary clip pad being a rod portion which is provided with a male fastener at a suitable place;

a secondary clip pad, the upper end of which is formed with a pad body opposite to the upper end of the above clip portion, said secondary clip pad being provided with a female fastener correspondingly provided for the male fastener such that both can fit together to form a rotation fulcrum at the fitting joint, then the primary clip pad and the secondary clip pad being associated with each other;

an elastic element being disposed between the primary clip pad and the secondary clip pad, both ends of the elastic element being respectively abutted against suitable spots of the primary clip pad and the secondary clip pad such that the clip portion of the primary clip pad and the pad body of the secondary clip pad can be clamped together or separated away by the recovery principle of the elastic element;

a pad block, being a flexible non-insulator which can transmit electric current of suitable value according to the control concerned, said pad block being disposed within the above accommodation space, an abutting surface corresponding to said pad body being formed on the upper side of the pad block and a clipping groove being formed at the side edge, such that one end of a lead wire of the electrode can be clipped in the clipping groove of the pad block; when a pair of opposite force is applied to cause the bottom ends of the primary clip pad and the secondary clip pad to be closed together, the primary clip pad and the secondary clip pad move relative to each other with the rotation fulcrum as the rotation center so as to separate the clip portion of the primary clip pad, the abutting surface of the pad block, and the pad body of the secondary clip pad, simultaneously the elastic element is compressed so that the abutting surface and the pad body can be in separated state; when the force pair applied on the primary clip pad and the secondary clip pad is released, the primary clip pad and the secondary clip pad conduct relative motion with the rotation fulcrum as the rotation center so as to allow the clip portion of the primary clip pad, the abutting surface of the pad block, and the pad body of the secondary clip pad to come close together, simultaneously the elastic element is recovered so that the abutting surface and the pad body can clamp at any place of the ear in face contact manner.

Another Taiwanese Patent No. M277466 entitled “ Electrode Pad ” discloses another type of electric therapy device which is electrically connected to an external electric therapeutic device for providing electric stimulation, and which is communicated with an air-extracting device. The electric pad comprises:

a reverse cup shape body which defines a suction space with its opening facing downward, and which has a through hole penetrating said body and communicating with said suction space, the through hole being communicating with the air-extracting device so as to suck the air within the suction space;

a conductive pad having a contact portion provided to encircle around the bottom edge of said body, and a conduction portion extending from the contact portion to the through hole so as to be electrically connected to the external electric therapeutic device.

Another Taiwanese Patent No. M 307395 entitled “ Pad for Sore Relief and Stress Reduction” discloses another type of electric therapy device. The pad body is made by silicone rubber mixed with far infrared material. The bottom surface of the pad body is a flat surface, while the top surface is approximately a convex shape. A recess is provided at the center of the bottom surface for the attachment of germanium particle. A temperature strip is provided closely to the outer edge of the pad body, and an air permeable structure is also provided on the pad body. In this manner, the pad body is adhered on the sore spot or acupuncture points of users by means of air permeable adhesive tape. By the cooperative action of the free electrons with negative charge (minus ion) of germanium and the far infrared, radicals carrying positive ion formed on the sore spot can be neutralized through skin so as to mitigate sore, ache or pain and to adjust biocurrent.

Further, another Taiwanese Patent No. M 326831 entitled “ Dual Energy Health Pad ” discloses another type of electric therapy device which comprises a seat body, a magnet and a ceramic component, wherein:

said seat body has a groove the size of which is just equal to that of the magnet such that the magnet can be accommodated within the groove;

the magnet is disposed within the groove of the seat body and is fixed therein by adhesive;

said ceramic component is disposed above the magnet and is fixed thereon by adhesive.

However, the above electric therapy devices have the following disadvantages during practical application:

1. The above electric therapy devices are fixed on the body portion by pad, clip or are directly carried on the body portion to conduct therapy. However, the lifetime is limited by durable time, for example, pad type is limited by numbers of use, while clip type often causes elastic fatigue after long time usage or causes uncomfortableness of users due to overtightness of spring newly used.

2. The pads used in the pad type electric therapy device are often discarded after usage, while the clips in the clip type electric therapy device should be replaced when spring suffers elastic fatigue after long time usage. Thus, these devices quite deviate from the appeal of green environmental protection currently prevailing in the world.

### SUMMARY OF INVENTION

In view of above disadvantages, this invention provides a magnetic clip for ear acupuncture points which has magnetic bodies provided therein. Magnetic therapy action can be conducted on ear acupuncture points by magnetic force generated from the magnetic bodies, and electric current can also be drawn into the magnetic bodies to conduct electric therapy through a stimulating probe so that dual physical therapy effect containing magnetic therapy and electric therapy can be obtained. In addition, the magnetic bodies can provide the required holding force so as to avoid drop-down of the clip. As the conventional pad or clip is substituted by the inventive magnetic clip, the cost is low. Also, enhancement on the comfortableness and convenience for users can be achieved during therapy conducted on ear acupuncture points.

A preferred embodiment of the magnetic clip for ear acupuncture points of the present invention is characterized in that it essentially comprises a first clip body and a second clip body. The first clip body is a disk shape body having an insulation cap provided on one side, a first magnetic body being accommodated between the first clip body and the insulation cap and being coupled with a lead wire which in turn is connected to an electric stimulator, a stimulating probe being provided at the center of the other side of the insulation cap for abutting with the first magnetic body, so as to conduct electrically or magnetically stimulating therapy on ear acupuncture points. The second clip body has a second magnetic body which is coupled with a lead wire.

According to the preferred embodiment of the magnetic clip for ear acupuncture points of the present invention, the first clip body and the second clip body are disk type insulators.

### BRIEF DESCRIPTION OF THE ACCOMPANYING DRAWINGS

FIG. 1 is a perspective exploded view showing the magnetic clip for ear acupuncture points of the present invention.

FIG. 2 is a perspective assembled view showing the magnetic clip for ear acupuncture points of the present invention.

FIG. 3 is a view of the magnetic clip for ear acupuncture points of the present invention during use.

### DETAILED DESCRIPTION OF PREFERRED EMBODIMENT

The technical content, objects and effectiveness of the present invention will become more apparent by the detailed description of a preferred embodiment of the present invention in conjunction with the accompanying drawings.

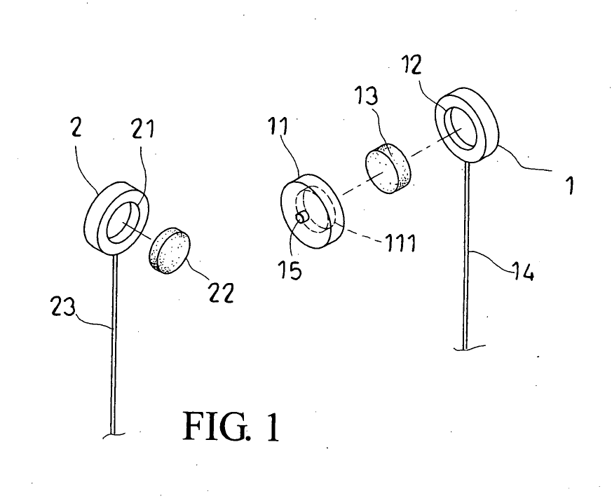

Referring to FIG. 1, the magnetic clip for ear acupuncture points according to the present invention comprises a first clip body (1) and a second clip body (2), in which:

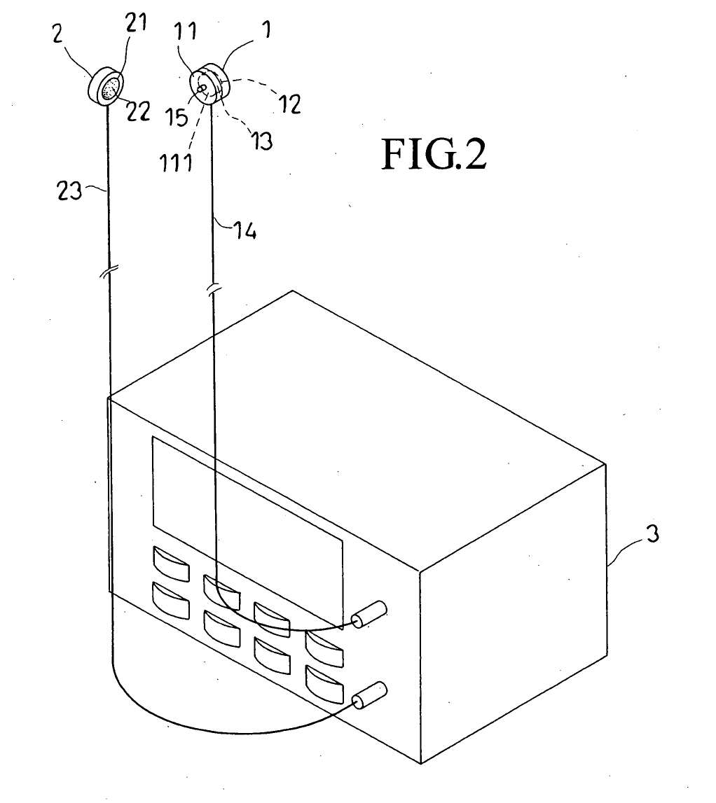

the first clip body (1) is a disk shape insulator which has an insulation cap (11) provided on one side. Cavities (12), (111) opposite to each other are respectively provided on the first clip body (1) and the insulation cap (11). A first magnetic body (13) is accommodated within the opposite cavities (12), (111) and is coupled with a lead wire (14) so as to be connected to an electric stimulator (3), as shown in FIG. 2. Further, a stimulating probe (15) is provided at the center of the other side on the insulation cap (11), which is abutted with-the first magnetic body (13) so as to conduct electrically or magnetically stimulating therapy on ear acupuncture points;

the second clip body (2) is also a disk shape insulator. A cavity (21) is provided on one side of the second clip body (2), in which a second magnetic body (22) is accommodated. Similarly, a lead wire (23) is coupled with the second magnetic body (22).

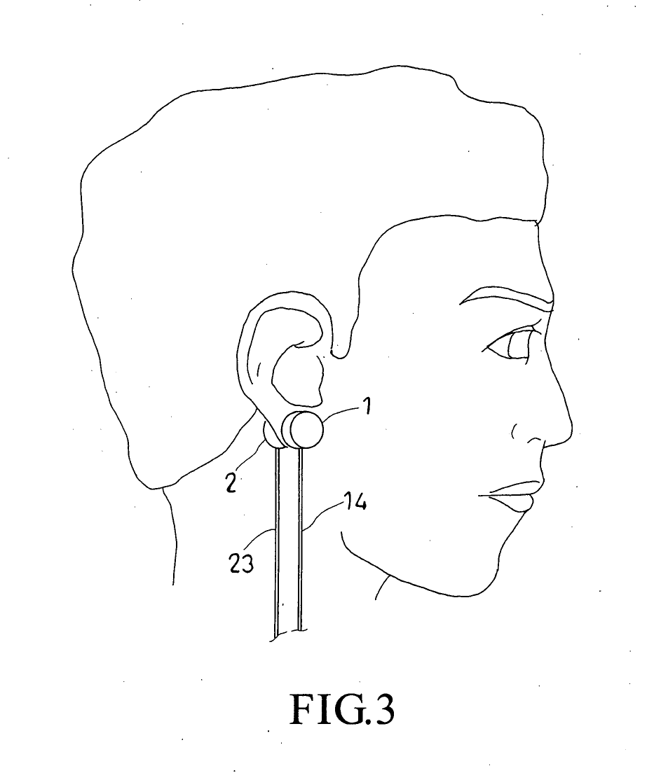

During use, as shown in FIGS. 2 and 3, the first clip body (1) is firstly placed at the front side of the ear with the stimulating probe (15) directing toward certain acupuncture point, and the second clip body (2) is disposed at the rear side of the ear. Thus, the first magnetic body (13) of the first clip body (1) and the second magnetic body (22) of the second clip body (2) are attracted with each other by magnetic force so as to sandwich the ear between them. The magnetic force generated between the first magnetic body (13) and the second magnetic body (22) can conduct magnetic therapy action upon ear acupuncture points. Simultaneously, the electric stimulator (3) can be started to send electric current to the first magnetic body (13) and the second magnetic body (22) respectively through the lead wires (14), (23), so as to conduct electric therapy via the stimulating probe (15). In this manner, dual physical therapy effect containing both magnetic and electric therapy on ear points can be achieved simultaneously.

However, the above embodiment or drawings are not intended to restrict the structural aspect, fixing structure or usage of the present invention. Variations of modifications made by the person having ordinary skill in the art without departing from the spirit and scope of the present invention are still regarded to be within the scope of the present invention.

Based on the foregoing, the magnetic clip for ear acupuncture points of the present invention has the following advantages when comparing with the conventional structure.

1. The magnetic bodies of the present invention can conduct magnetic therapy effect on the ear points; simultaneously the stimulating current can be drawn into the magnetic bodies to conduct electrical therapy through the stimulating probe. In this manner, dual physical therapy effect containing both magnetic and electric therapy on ear points can be achieved simultaneously.

2. The magnetic bodies of the present invention can provide the required holding force so as to avoid the drop-down of them. Also, the cost is low and enhancement on the comfortableness and convenience for users can be achieved during ear points therapy.

3. The magnetic clip for ear acupuncture points of the present invention is easy for hand-carry, and the insulation plate is easy to replace. Therefore, it is extremely convenient in practical use.

4. The magnetic clip for ear acupuncture points of the present invention can be used for a long time, and hence it is not necessary to worry about the problem of phase-out due to degeneration in the course of time. Thus, this invention reduces the depletion of resource, hence it matches green concept of environmental protection.

Summing up above, the embodiment of the present invention is surely able to reach expected effectiveness in application, and the specific structure disclosed herein is not only unprecedented in the same category of product but also not open to the public before application.

What is claimed is:

1. A magnetic clip for ear acupuncture points, comprising:
a first clip body having an insulation cap provided on one side, a first magnetic body being accommodated between the first clip body and the insulation cap and being coupled with a lead wire, a stimulating probe being provided at the other side of the insulation cap for abutting with the first magnetic body;
a second clip body having a second magnetic body provided therein which is coupled with a lead wire.

2. A magnetic clip for ear acupuncture points as claimed in claim 1, wherein the lead wires of said first magnetic body and said second magnetic body are coupled to an electric stimulator.

3. A magnetic clip for ear acupuncture points as claimed in claim 2, wherein said first clip body and said second clip body are both disk type.

4. A magnetic clip for ear acupuncture points as claimed in claim 2, wherein said first clip body and said second clip body are both insulators.

5. A magnetic clip for ear acupuncture points as claimed in claim 2, wherein said first clip body and said insulation cap are provided with cavities opposite to each other, and said first magnetic clip is accommodated within the space formed by the opposite cavities.

6. A magnetic clip for ear acupuncture points as claimed in claim -2, wherein said second clip body is provided with a cavity in which said second magnetic clip is provided.

7. A magnetic clip for ear acupuncture points as claimed in claim 1, wherein said first clip body and said second clip body are both disk type.

8. A magnetic clip for ear acupuncture points as claimed in claim 1, wherein said first clip body and said second clip body are both insulators.

9. A magnetic clip for ear acupuncture points as claimed in claim 1, wherein said first clip body and said insulation cap are provided with cavities opposite to each other, and said first magnetic clip is accommodated within the space formed by the opposite cavities.

10. A magnetic clip for ear acupuncture points as claimed in claim 1, wherein said second clip body is provided with a cavity in which said second magnetic clip is provided.
